(19) United States
(12) Patent Application Publication
(10) Pub. No.: US 2025/0380901 A1
(43) Pub. Date: Dec. 18, 2025

(54) DEVICE FOR GENERATING A SEQUENCE OF IMAGES REPRESENTING A CONTINUOUS SERIES OF MOVING BONE ELEMENTS

(71) Applicant: YNNOV, Antibes (FR)

(72) Inventor: Yves STEPHAN, ANTIBES (FR)

(21) Appl. No.: 18/876,442

(22) PCT Filed: Jun. 19, 2023
(86) PCT No.: PCT/EP2023/066487

§ 371 Date: Dec. 18, 2024

(30) Foreign Application Priority Data
Jun. 20, 2022 (FR) FR2206056

(51) Int. Cl. A61B 5/00; A61B 5/11; G06T 13/40

(52) U.S. Cl. CPC A61B 5/4566; A61B 5/1122; A61B 5/6833; A61B 5/725; A61B 5/7435; A61B 5/749; G06T 13/40; A61B 2562/0219; A61B 2562/028; G06T 2207/30012

(57) ABSTRACT

A device for generating a sequence of images configured to represent in 3D a continuous series of aligned moving bone elements belonging to a subject. The device includes a plurality of inertial units intended to be arranged facing a plurality of measuring points, chosen by a practitioner, on the continuous series of aligned bone elements of the subject to cyclically take measurements. It also includes at least one first treatment unit configured to receive the measurement data from the respective inertial units and transform them into orientations of the inertial units. The device further includes a second treatment unit configured to receive the orientations of the inertial units; to update the orientations and the positions of the measuring points facing the inertial units on the basis of the orientations of the inertial units as and when the second treatment unit receives them; and then to form a curve representing the continuous series of bone elements using the positions of the thus-obtained measuring points and the interpolations between the positions of the measuring points. The second treatment unit is configured to allow the practitioner to input anatomical data or observation data used to correct the curve. The device is used to reconstruct a realistic image of a continuous series of aligned moving bone elements on the basis of a minimum amount of data. The device is used to establish reliable diagnoses from the obtained images and remotely assist a patient with exercises.

### TECHNICAL FIELD

The invention relates to the field of medical imaging devices. More precisely, it relates to imaging devices for examining a continuous succession of moving bone elements, and even more precisely to devices producing sequences of images of a succession of moving bone elements.

### TECHNOLOGICAL BACKGROUND

Bipedalism is a result of the evolution of human anatomy. This verticalization has as corollary the need to maintain a posture and to manage movement. This introduces, in addition to a suitable complex anatomy, the notion of mechanisms for managing these postures and movements with a strongly implied notion of energy saving both in posture maintenance and in the movement function. This is the field of functional biomechanics in particular of the supporting column and more precisely of the spinal column.

The study of posture and movement of the entirety and of some of the elements contributing thereto is essential to better comprehension, in particular in case of dysfunction.

Specifically, all or some of this entirety is the seat of dysfunctions that are the origin of disorders that here are grouped under the term postural disorders or disorders of mobility of the spine. These disorders include postural abnormalities and movement abnormalities.

These disorders are very common and on the increase. They are considered in France to be a national health issue, as they are responsible for major health and disability expenditures.

Optimization of treatment of these disorders requires the ability to support the therapeutic strategy and to track progression in particular based on a functional analysis of posture and/or performance of movements.

Optimization of patient treatment requires a good understanding of the patient, in particular in the case of physiotherapy.

In case of rehabilitation, for it to be optimal, it is necessary to have real-time and functionally accurate control of the part the entirety or elements of which are to be tracked. This is also true for sports training inter alia.

It should be noted that professionals are increasingly employing hands-free devices allowing them to treat a number of patients simultaneously.

Optimization of the transmission of information between the various actors involved in a course of treatment of such disorders

The problem is how to make available to the various users requiring it, a device allowing:
on the one hand, an analysis of posture and movement in all planes based on a precise display of modelling of the studied part “in 3D” and “in real time”;
on the other hand, a didactic medium allowing comprehension by the patient of the dysfunction that she or he has and of the sought objective;

on the other hand, a functionally accurate medium permitting coaching or monitoring of the patient during her or his exercises.

It is further necessary for it to work regardless of the position of the patient.

It is further necessary for these devices to be easy and not very time-consuming to use, space-saving, and financially accessible to as many people as possible so as to form part of a basic medical arsenal.

However, the various current procedures do not meet all of these needs

X-ray based systems (standard full-body x-ray) allow vertical posture analysis but not analysis of movement. They do not allow movement to be analyzed, and even less so in 3D. They are bulky, expensive and do not meet most of the required criteria.

CAT scans and MRI are unsuitable for analysis of posture or movement.

The EOS system allows analysis of vertical posture in 3D, but neither analysis of movement nor real-time analysis. It is bulky and expensive.

The systems are camera-based systems. Single-camera systems such as SAM 3D allow neither study in 3D nor movement.

Multi-camera systems allow analysis of movement in 3D and in real time. However, they do not allow all the desired positions, and are bulky, of a complexity requiring specification, expensive and unsuitable to current practice.

It has also been proposed to capture the movements of the patient via inertial measurement units worn by the patient. Inertial measurement units are electronic components that measure linear accelerations on three axes and velocities of rotation about these three axes.

Products of this type are available at the current time.

For example, the product marketed under the brand name Bioval (registered trademark) uses up to 4 inertial measurement units but no more. It allows an analysis of movement but only in one plane at a time, requiring the movement to be repeated and making it impossible to analyze a movement in its 3 planes. It does not make provision for intuitive display of the studied part but merely for simultaneous plots of the progression of angulations over time. It makes provision neither to monitor nor to coach exercises.

Another example is the product marketed under the trademark Truposture (registered trademark). It is a question of a T-shirt comprising four “daughter” circuit boards and one “mother” circuit board that drives the “daughter” circuit boards. Each of the circuit boards comprises one inertial measurement unit. The inertial measurement units are placed along the spinal column. The motherboard in addition comprises a Bluetooth® communication module and an alarm. The Truposture T-shirt analyzes the position of the back in a sagittal plane and emits an audible alarm if the position is “wrong”. It is insufficiently accurate.

### SUMMARY

One aim of the invention is to provide a device producing a sequence of images in the three dimensions of space, of a continuous succession of aligned bone elements of a skeleton, the succession moving, the skeleton belonging to a subject under observation, who is a living vertebral being, for the purpose of medical examination or rehabilitation support. Another aim of the invention is to minimize the information recorded in order to make the device suitable for the technical means generally available in a medical practice. Another aim of the invention is to allow the practitioner to modify the “inputs” of the device until the representation of the succession of bone elements satisfies her or him. One idea behind the invention is to measure quantities based on which it will be possible to deduce a representation of the movement of the bone elements through geometric rotations. Another idea behind the invention is to take measurements only at a small number of points. Another idea behind the invention is to compose the images of the sequence on the basis of the positions of the measurement points, of interpolations between the positions of the measurement points and of data input by the practitioner. Another idea behind the invention is to calculate interpolations between the observations and to display the corresponding image before proceeding to the following measurements, in “real time”.

Below, the following terms will be employed with the following definitions.

Image sequence: images that are concatenated at regular intervals. In the context of the invention, the images reproduce a single scene, that of a succession of moving bone elements. Each image reproduces the succession of bone elements at a given time; thus the image sequence reproduces a sequence of positions of the succession of bone elements and gives the impression of a movement as a result of a flipbook effect.

Continuous succession of aligned bone elements: ordered set of bone elements in which each bone element is articulated with the next one. It is the articulations that give the succession its continuous character. The successions of bone elements in question form lines as in the spinal column or lower limbs.

Facing: an inertial measurement unit of the device of the invention is said to be “facing” a bone element when it is able to capture the movement thereof, possibly through an intermediate biological tissue such as the skin.

Selected: a user of the device of the invention must beforehand select a certain number of measurement points facing which she or he will arrange inertial measurement units (one per measurement point).

Order: a user of the device of the invention must, once she or he has placed the inertial measurement units on the continuous succession of bone elements, designate the first bone element and in what direction the line of bone elements should be followed to find the next inertial measurement unit after a given inertial measurement unit.

First processing unit: unit for processing the digital signal generated by the inertial measurement units. In the invention, the function of the one or more first processing units is essentially to convert the measurements of velocity of rotation produced by the inertial measurement units into a rotation in space indicating an orientation of the facing bone element.

Axis/center of rotation: axis/center about which the continuous succession of bone elements that the invention allows to examine rotates.

Real time: the device of the invention operates in real time in the sense that between two successive cycles of measurements taken by the inertial measurement units, it completes a complete processing cycle in the first and second processing units, including display of the produced curve, before the next measurement.

The terms “inertial measurement unit” and “sensor” are interchangeable below and will be used to mean the same thing.

In one embodiment, the invention provides a device for generating an image sequence, said device being configured to represent, in the three dimensions of space, at least one continuous succession of aligned bone elements of a skeleton, said at least one succession being able to be moving, the skeleton belonging to a subject, the device comprising:
a plurality of inertial measurement units,
   each inertial measurement unit being configured to cyclically measure velocities of rotation about three axes associated with the inertial measurement unit, thus forming measurement data,
   the inertial measurement units being intended to be placed facing selected measurement points on the succession of bone elements, in an amount of one inertial measurement unit per measurement point, said measurement points thus forming a plurality of measurement points to be ordered
at least one first processing unit, configured to:
   receive the measurement data of the plurality of inertial measurement units,
   upon receipt of new measurement data, update based on said measurement data an orientation of each of the inertial measurement units of the plurality of inertial measurement units,
and a second processing unit, configured to
   receive the orientations of inertial measurement units from the at least one first processing unit,
   upon receipt of new orientations of inertial measurement units,
      for each measurement point of the plurality of measurement points, update:
         an orientation, based on the received orientations of inertial measurement units;
         except for the first measurement point, a position, based on the orientation of the updated previous measurement point and on a table of distances between the bone elements of the succession of bone elements;
      the position of the first measurement point being determined based on an axis of rotation of the continuous succession of bone elements
      re-interpolate, for each pair of successive measurement points of the plurality of measurement points, between the measurement points of said pair, a curve segment;
      re-form a curve by joining the curve segments.

By virtue of these features, the device makes it possible to obtain, via measurement or interpolation, sets of points representative of a spinal column in space, which, in sequence, will produce a relief image of the moving spinal column. Digitization makes it possible to represent the movements of the vertebrae by means of geometric rotations, this allowing the volume of observation data to be considerably reduced. It makes it possible to partly construct the curve representing the spinal column by interpolation, and thus to reduce the number of points and therefore the volume of digital data to be recorded. The user matches the curve with the ground truth that it is supposed to represent.

According to embodiments, such a device may have one or more of the following features.

According to one embodiment, the second processing unit is configured to, for each inertial measurement unit, indicating a direction assumed to be Earth's magnetic north,
calculate a divergence correction rotation, said divergence correction rotation converting a reference direction common to the inertial measurement units into the direction indicated by the inertial measurement unit;
when the orientation of the inertial measurement unit is updated, amend the orientation of the inertial measurement unit to an orientation with respect to the reference direction via right composition by the respective divergence correction rotation.

Thus, the device of the invention makes it possible to carry out a sort of calibration of the inertial measurement units to a common standard.

According to one embodiment, the invention provides a device for generating an image sequence, said device being configured to represent, in the three dimensions of space, a moving spinal column belonging to a subject, the device comprising:
a plurality of inertial measurement units,
   each inertial measurement unit being configured to cyclically measure velocities of rotation about three axes associated with the inertial measurement unit, thus forming measurement data,
   the inertial measurement units being intended to be placed facing selected vertebrae of the spinal column, in an amount of one inertial measurement unit per vertebra, said vertebrae thus forming a plurality of vertebrae to be ordered
at least one first processing unit, configured to
   receive the measurement data of the one or more inertial measurement units, respectively,
   upon receipt of new measurement data, update based on said measurement data, an orientation of each of the inertial measurement units of the plurality of inertial measurement units,
and a second processing unit, configured to
   receive the orientations of inertial measurement units from the at least one first processing unit,
   upon receipt of new orientations of inertial measurement units,
      for each vertebra of the plurality of vertebrae, update a vertebral orientation, based on the received orientations of inertial measurement units;
         except for the first vertebra, a position, based on the vertebral orientation of the updated previous vertebra and on a table of distances between the vertebrae of the spinal column;
      the position of the first vertebra being determined based on an axis of rotation of the continuous succession of bone elements,
      thus forming a position of the plurality of vertebrae;
      re-interpolate, for each pair of successive vertebrae of the plurality of vertebrae, between the vertebrae of said pair, a curve segment;
      re-form a curve by joining the curve segments.

According to one embodiment, the device of the invention has available to it a register that contains, for each pair of vertebrae of the spinal column of the subject, a ratio of the distance of said vertebrae to the length of the spinal column of the subject; and for each pair of vertebrae of the spinal column of the subject, the distance separating the vertebrae of said pair being calculated by multiplying the corresponding ratio of the register by the length of the spinal column of the subject; and for each pair of vertebrae of the spinal column of the subject, the distance separating the vertebrae of said pair being calculated by multiplying the corresponding ratio of the register by the length of the spinal column of the subject; and said distance being automatically entered into the table of distances between the vertebrae.

Thus the obtained curve respects an anatomical law and more faithfully reproduces a spinal column.

In one embodiment, the second processing unit is configured to allow a user to modify the table of distances.

In one embodiment, the second processing unit is configured to:
for each vertebra of the plurality of vertebrae and/or for the head, when an inertial measurement unit with an inertial measurement unit orientation is placed on the respective vertebra or head, which have a vertebral orientation, calculate a correction rotation, the correction rotation converting the orientation of the inertial measurement unit into a vertebral orientation of the respective vertebra or head;
upon receipt of new measurement data, for each vertebra of the plurality of vertebrae and/or for the head, prior to updating the orientations of the vertebrae and/or head, correct the orientation of the respective inertial measurement unit via application of the calculated correction rotation.

Thus, errors in the orientation of the inertial measurement unit resulting from a difficulty in rigorously placing it where necessary on the spinal column or from instability of the electronic signal generated by the inertial measurement units may be corrected. However, an inertial measurement unit intended for the skull may also be placed anywhere on the skull.

In one embodiment, the second processing unit is configured to allow a user to modify the correction rotations of the vertebrae.

According to one embodiment, the device of the invention comprises a database.

According to one embodiment, the device of the invention comprises a display unit.

According to one embodiment, the device of the invention further comprises a database and/or a display unit, and the second processing unit is configured to allow a user to trigger, in each measurement cycle:
recording of the position of the plurality of generalized vertebrae then formed in the database;
and/or display of the curve then formed on the display unit.

Thus, the practitioner may reduce the number of positions recorded to the strict minimum required to meet her or his needs.

According to one embodiment, the measurement frequency is adjustable by a user.

In one embodiment, the second processing unit is configured to:
allow a user to retrieve from the database at least one position of a second plurality of vertebrae; and, for each position of the second plurality of vertebrae:
   interpolate between the vertebrae of each pair of successive vertebrae of the second plurality of vertebrae, a second curve segment; and
   form a respective second curve by joining the second curve segments; and in addition configured to display the second curves on the display unit.

In one embodiment, the second processing unit is configured to, when the second processing unit receives new orientations, monitor whether the position of the updated plurality of vertebrae is in a vicinity of the position of the second plurality of vertebrae; and, if so, to generate a signal specific to each position of the second plurality of vertebrae.

According to one embodiment, the second processing unit is equipped with a first pedal for recording the positions of the plurality of vertebrae and with a second pedal for stopping recording the positions of the plurality of vertebrae; and/or with a voice command combining the functions of the first and second pedals.

### Synchronized Sensor Alignment

According to one embodiment, the device according to the invention further comprises a system for synchronously initializing the sensors or inertial measurement units. This makes it possible to avoid asynchronous initialization of the sensors, responsible for generation of different data from the outset.

This system may comprise:
definition then declaration of sensors considered to form part of a set,
optionally, the sensors themselves,
a housing containing the sensors during the initialization phase and imposing thereon a common orientation in the 3 planes, and
an angular correction algorithm configured in such a way that the initial data generated by each sensor are individually corrected by a correction angle such that their calculated angular position is identical.

These correction angles are advantageously saved and applied until a new alignment.

### Reference Position

It is also advantageous to compensate for drift inherent to the electronics of the

sensors. According to one embodiment, the device according to the invention is configured to implement an algorithm such that:
at the beginning of the examination, when drift (which increases with time) has not yet occurred, the initialization procedures having been performed, the sensors being arranged on the patient and the necessary angular corrections having been made, all the information allowing modelling corresponding to the patient adopting a reproducible “reference position” is recorded, and
at any time during the examination, when the patient is instructed to return to this reference position, a function calculates the angular adjustments necessary for juxtaposition of the instantaneous position and the so-called base position.

These angular adjustments are advantageously retained until a new correction is made.

### Pairing the Position of the Arranged Sensors and the Positions Defined for Modelling Purposes

According to one embodiment, the device according to the invention is configured such that:
the positions of the sensors are paired, the sensors being positioned freely on the patient at the discretion of the user and facing identified bone elements, these positions being communicated,
the calculation of the distances between two sensors, essential to correct modelling, is carried out in light of a pre-filled table collating the relative distances between each neighboring bone element,
the calculations of the modelling include the location of the sensors and the distance separating them, and
in the event of a positioning level error, the latter is corrected by modifying this pairing.

These steps allow information on the distance between the sensors to be communicated, correct correlation between the position of the sensors on the patient and the model and judicious positioning of the sensors on the patient.

### Angular Correction of the Data Generated by the Sensors

In order to be able to correctly represent the orientation of a bone element, the sensor must advantageously be positioned in such a way as to respect the defined axis employed in the modelling of this bone element. However, it may not be straightforward to position the sensor in this way, and it may be necessary to compensate for poor positioning.

According to one embodiment, the device according to the invention is configured in such a way that an angular correction is applied to the angular data generated by the sensors, this correction being stored and taken into account in the calculations leading to the modelling.

A set of cursors may apply in the three planes separately an angular correction to the data generated by each sensor. The correction may be immediately visualized in the form returned by the modelling.

In one variant, a first assistance function is provided, which makes use of previously defined positional data.

In the particular case of the head, correctly positioning a sensor on the head is not simple. In biomechanics the head is defined to be horizontal when the patient's line of sight is horizontal. An automatic angular correction may be made to the data generated by the sensor, which may then be positioned anywhere on the head.

### Automatic Angular Adjustment in Light of Data Communicated Beforehand

Anatomical or positional data may be communicated prior to an examination.

These angular data may be provided by the user, but principally result from radiological examinations (conventional x-rays, EOS, etc.).

According to one embodiment, the device according to the invention is configured to make the angular adjustments automatically, in order to conform to these data, referred to as anatomical or positional data, communicated beforehand.

Once the sensors have been arranged on the patient and how they are positioned has been communicated, the model may be fitted to the recorded positional data, provided that the patient adopts the same position. This is generally the erect position, facing straight ahead.

The recorded data may be: sacral slope, lumbar lordosis, dorsal kyphosis, or any other angle usable for an angular correction.

Thus, the modelling of the patient's spinal column will be performed in real time. The corrections made are recorded.

### Theoretical Modelling of Data Communicated Beforehand and Automatic Angular Adjustment

According to one embodiment, the device according to the invention is configured to perform the following steps:

In a first step, anatomical and positional data resulting from radiological examinations (such as conventional x-rays, EOS, inter alia) are communicated, these data in particular including data resulting from x-rays such as:
in the sagittal plane, the sacral slope then a continuity of adjacent angles, which will either be normal, L5-L1 lordosis, T12-T1 kyphosis, C7-C1 lordosis, etc.,
in the frontal plane, if necessary, so-called Cobb angles of lateral inclination, limiting a set of vertebrae (for example Cobb angle between T4 and T12), and
data in the coronal plane.

In a second step, these data are used in the calculations leading to creation of a model, referred to as the static 3D theoretical model, of the spinal column and pelvis in their entirety.

In a third step, although the angular information used relates to groups of vertebrae or other bone elements, the calculations of this theoretical model establish and record in the patient's file angular data on each bone element individually, and in particular on each vertebra, the head, each part of the pelvis, and the lower limbs.

In a fourth step, an automatic angular adjustment of the real-time model of the patient is carried out in light of the data of said theoretical model. During an examination, the sensors being initialized then arranged on the patient, who adopts a posture identical to the one adopted during the radiological examination, an angular correction of each representation of the bone elements may be made to fit the real-time modelling of the patient to the recorded theoretical model.

Since the creation of the theoretical model results in angular data on each bone element being recorded, the positioning level of the sensors on the patient is advantageously freely chosen and does not require a placement corresponding to the angles used to create the theoretical model.

This automatic adjustment may be re-made at any time during the examination, the patient repositioning her or himself under the same conditions if necessary, in particular in the event of electronic drift, of change of sensors, or of any abnormality.

### Modelling Integrating the Pelvis and the Biomechanics of the Pelvo-Sacro- Lumbar Complex

Conventionally, various angular calculations made based on radiological examinations may be recorded, and among these, some may concern the pelvis. They in particular correspond to:
pelvic parameters that are referred to as anatomical: pelvic incidence, distance between the hip joints, pelvic thickness; these parameters affect the biomechanics of the pelvo-sacro-lumbar complex, and/or
pelvic parameters that are referred to as positional: sacral slope and pelvic tilt.

As known, the parameters pelvic incidence, sacral slope and pelvic tilt are related by an equation. These parameters vary with the posture of the patient. Sacral slope corresponds to the angle made between the sacral endplate and the horizontal. Because the inclination of the last lumbar vertebra is dependent thereon, the posture of the entire suprajacent spine is dependent thereon. It is therefore essential to determine sacral slope angle as accurately as possible. This angle could be deduced from angular data delivered by a sensor arranged facing the posterior surface of the sacrum and more particularly the second sacral vertebra, the theoretical point of rotation of the sacrum, but since sacral anatomy varies, this angular information will not allow sacral slope angle to be deduced reliably.

In order to overcome these drawbacks, according to one embodiment, the device according to the invention is configured to:
communicate pelvic parameters resulting from radiology,
model the pelvis via integration into the calculations, in view of reliable modelling of the pelvis, of anatomical parameters,
determine the instantaneous center of rotation of the sacrum; the real-time modelling potentially integrating rotation of the sacrum about the line between the center of rotation and the second sacral vertebra, as well as the movement of the entire suprajacent column as deduced from data generated by sensors arranged on the vertebrae, and
determine a reliable sacral slope angle in the erect position using an angular adjustment as seen above of the sensor arranged on the sacrum depending on the sacral slope calculated during a radiological examination in the same posture.

### Modelling Integrating the Lower Limbs

By virtue of the invention, the spine (extended to the head) considered as a continuous succession of moving bone elements is modelled, and the pelvis and the biomechanics of the pelvo-sacro-lumbar complex are incorporated. The pelvis, including the hip joints, may be modelled, the anatomy of the pelvis and its parameters (such as pelvic incidence, thickness and distance between the hip joints) being modellable in light of the parameters derived from the radiological data.

The whole of the column suprajacent the sacrum, including the sacrum, has as its axis of rotation during movements the instantaneous center of rotation of the sacrum. Said center is coincident with the center of the line joining the hip joints. Thus, the model of this whole advantageously has a principal point called the initial point around which the entire model moves.

The two lower limbs are advantageously modelled using the same process. Each is, just like the column, a continuous succession of moving bone elements. The femur, tibia and foot are considered to be non-deformable bone elements answering to this continuous succession of elements. A sensor arranged facing bone markings at each level is able to generate angular information relating to bone elements of the lower limbs.

The various functions described above are advantageously usable regardless of the type of succession of bone elements. In respect of lower limbs, information on the relative length of the various segments of the lower limbs may be used. In one variant, by default, a length referred to as the standard length is used.

Each model advantageously has a principal initial point about which the model of the lower limb moves. It may correspond to the femural head.

Each femural head is advantageously coincident with the hip joint of the model of the head-spine-pelvis complex.

Thus, these three continuous successions of moving bone elements are connected and form a whole the initial overall center of which is the instantaneous center of rotation of the sacrum.

### The Need for Measurement: Angle Tracking

Having achieved display in real time and in three planes of a model representing with fidelity the spine (extended to the head, pelvis and lower limbs), analysis of the dynamic behavior of this spine improves with the number of measurements, in particular angular measurements.

It may be useful to take measurements in a set position, with a view to analyzing an angular anomaly, but it would also be very useful to be able to track an anomaly during movement: how does a scoliosis or other disorder behave during movement, how does a particular area of a patient with arthrodesis behave, etc.

Although it is possible to measure a position referred to as set during a radiological examination, no known system allows the variation in an angle defined between two vertebrae or other bone elements to be tracked during a complex movement.

According to one embodiment, the device according to the invention is thus configured to:
define one or more angles such that the one or more sides of an angle are defined relative to one or more bone elements taken into account in the modelling, with:
   in the case where one side is relative to a bone element, the other side may be vertical or horizontal; if it is a question of representation of a vertebra, these angles advantageously correspond to angles of what is referred to as slope, when one of the sides is horizontal, and what is referred to as inclination, when one of the sides is vertical,
   in the case where the two sides of an angle are defined relative to two bone elements taken into account in the modelling, the definition of this angle advantageously induces tracking of a modification of curvature of the set of bone elements included in this angle; it may thus in particular be a question of tracking of modification of curvature of a set of vertebrae, for example angle of kyphosis, lordosis, scoliosis, the sides then corresponding to the tangents of the superior endplate of the selected superior vertebra and the inferior endplate of the selected inferior vertebra,
for a defined angle, perform the calculation of this angle in three planes and display it in real time during the movement, and collate the collected data relating to the angles.

### BRIEF DESCRIPTION OF THE FIGURES

The invention will be better understood, and other aims, details, features and advantages thereof will become more clearly apparent, from the following description of a number of particular embodiments of the invention, which are non-limiting and given merely by way of illustration, with reference to the appended drawings.

FIG. 1 shows a subject during examination by the device of the invention in a certain embodiment.

FIG. 2a shows a spinal column seen from the left in the sagittal plane.

FIG. 2b shows an extended spinal column seen from the left in the sagittal plane.

FIG. 3a shows a typical vertebra, from the left in the sagittal plane.

FIG. 3b shows the vertebra of FIG. 3a from above in the transverse plane.

FIG. 4a shows anatomical planes.

FIG. 4b shows anatomical axes that are suitably oriented for calculation of the Euler angles.

FIG. 5 shows an approximation of the curvature of the spinal column by a polygonal chain and a curve.

FIG. 6 shows a position of the spinal column during examination and recorded positions of the spinal column.

FIG. 7 shows a housing containing an inertial measurement unit, arranged on a vertebra through the skin and held in place by a patch.

FIG. 8 shows an inertial measurement unit arranged at an angle on the spinous process with respect to the desired position.

FIG. 9 shows distances between vertebrae.

FIG. 10 shows a human-machine interface making it possible to input anatomical data of the subject.

FIG. 11 shows a typical vertebra that has pivoted with respect to a neighboring vertebra.

FIG. 12 illustrates one example of angular adjustment.

FIG. 13 illustrates an example of generation of a theoretical model based on anatomical or positional data communicated beforehand and on automatic angular adjustment in light of this so-called theoretical posture.

### DESCRIPTION OF EMBODIMENTS

Although the invention was originally motivated by disorders of the back, the device 14 of the invention is applicable to any continuous succession of aligned bone elements of the skeleton 9. The device 14 is applicable, above all but not exclusively, to the arms, lower limbs and spinal column. The device 14 is also suitable for application to both arms and both lower limbs, with the spinal column if the user so desires, etc. The present application focuses on the case of the extended spinal column 9.

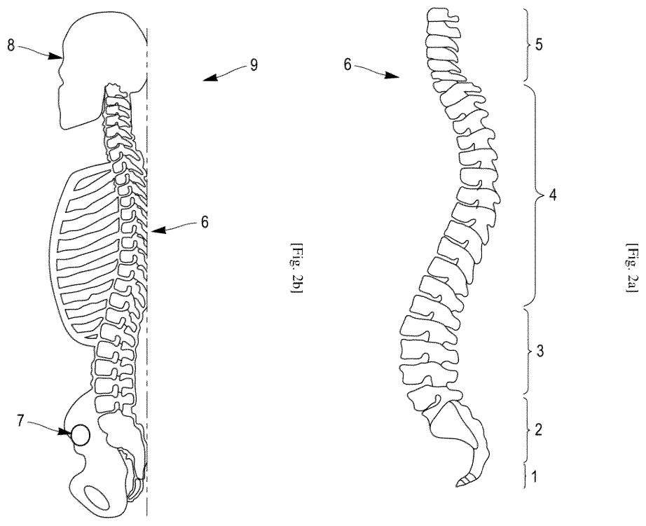

FIG. 2a shows a spinal column (or spine) 6 in its strictest sense, of a human being seen from the left. It comprises thirty-three vertebrae that are conventionally divided into sub-sets: seven cervical vertebrae 5, twelve thoracic or dorsal vertebrae 4, five lumbar vertebrae 3, five fused sacral vertebrae (the sacrum) 2 and four fused coccygeal vertebrae (the coccyx) 1. It is customary to number the vertebrae of a sub-set from top to bottom, assuming that the human being in question is standing; for example, C7 designates the 7^{th} cervical vertebra from the top.

FIG. 2b shows a spinal column 9 extended to the head 8. One of the advantages of the invention is that the head may be taken into account in diagnosis of disorders of the spinal column. The head has moreover been considered to be a vertebra by renowned specialists and called the “cephalic vertebra”.

There is a natural, anatomical order of the vertebrae (or two orders that exactly mirror each other) from the sacrum 2 to the head 8, making it possible to speak of the next or previous vertebra, or of a pair of successive vertebrae, etc.

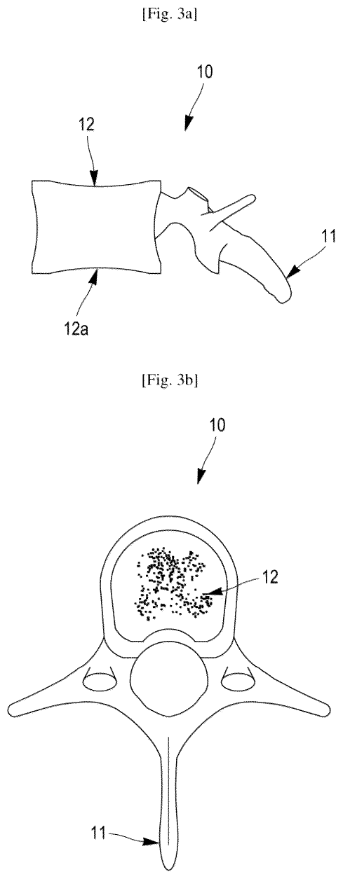

FIGS. 3a and 3b show a typical vertebra 10 (i.e. it could be any vertebra of the spinal column 6) seen from the left and from above, respectively. All the vertebrae of the spinal column 6 are similar in form, with the exception of a few details in respect of shape or size, justifying the notion of typical vertebra. A typical vertebra 10 comprises at the rear a spinous process 11, namely a pointy part that extends rearward and downward. It also comprises a superior endplate 12 and an inferior endplate 12a, which are almost flat and make contact with the neighboring vertebrae.

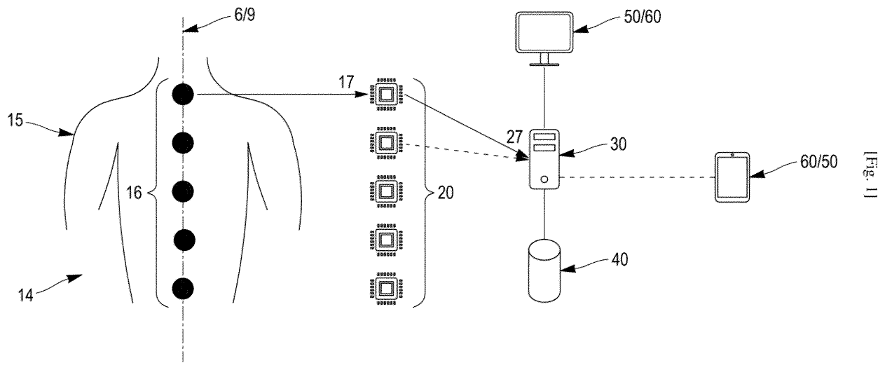

FIG. 1 shows a subject 15 under observation during examination by a device 14 according to the invention. The subject 15 shown is a human being, but it could be any vertebral being. A plurality of inertial measurement units 16 are placed facing vertebrae of the spinal column 6/9 of the subject, which has been represented by a dash-dotted line in FIG. 1. In the example shown, there are five inertial measurement units 16, but in variants that have not been shown, a larger or smaller number of inertial measurement units 16 may be provided as required. In order to limit the volume of measurements, it is preferable for the number of inertial measurement units 16 on the spinal column 6/9 to be limited, and in any case to be less than the number of vertebrae. The inventor has found that seven inertial measurement units 16 on the spinal column 9, including one on the head 8 and another on the sacrum 2, allow medical examinations of entirely satisfactory quality to be performed.

Prior to using the device, the practitioner must set an order of the inertial measurement units 16: the first inertial measurement unit 16, the second inertial measurement unit 16, etc. This makes it possible to speak of the next or previous inertial measurement unit, or of a pair of successive inertial measurement units, etc. The order of the inertial measurement units 16 must follow the natural order of the vertebrae, from the sacrum 2 to the head 8 or vice versa. The order of the inertial measurement units will then allow the measurement points to be ordered.

To make it easier to keep the inertial measurement unit 16 in a position as close as possible to the desired position, each inertial measurement unit 16 is contained, as shown in FIG. 7, in a small housing 80. The housing 80 is held against a vertebra by a flexible adhesive patch 81 that adheres to the housing and to the skin 13 around the housing.

The practitioner may advantageously choose an anatomical point as a fixed point referred to as the “principal center of rotation” 7, as indicated in FIG. 2b. The movement of the spinal column 6 will then be corrected using known mathematical methods. When the back or lower limbs are being examined, it is particularly advantageous to choose the middle of the line joining the hip joints as the principal center of rotation. This point is visible in common postures adopted by a person bending over or squatting, and it is of great clinical interest to observe it. However, the first inertial measurement unit 16 may also be chosen as principal center of rotation.

The device 14 also comprises a plurality 20 of first processing units, in an amount of one per inertial measurement unit 16. As a variant (not shown), there is one first processing unit 20 common to the plurality of inertial measurement units 16. The device 14 further comprises a second processing unit 30 connected, in the embodiment shown, to a database 40 and to one or more display units 50.

The second processing unit 30 is located remote from the subject 7 physically. The second processing unit 30 could be a personal computer.

The hardware elements placed on the back of the subject 15 and the second processing unit 30 communicate via a wired or wireless link (for example a Bluetooth® or Wi-Fi® link and preferably a Bluetooth Low Energy link (registered under the trademark Bluetooth Smart®).

It is necessary, prior to using the device 14, to obtain the curvilinear distances (referred to simply as “distances” in the rest of the application) between the inertial measurement units 16 and to input them into the second processing unit 30. The distance between the inertial measurement units 16 is none other than the distance between the facing vertebrae.

There are all kinds of methods for obtaining these distances. It is for example possible to use a measuring wheel moved along the spinal column 9 of a subject 15 beside the inertial measurement units 16. Another method consists in arranging a tape measure on the back of the subject 15 before putting the inertial measurement units 16 in place.

The device 14 also makes it possible, in one advantageous variant, to help the practitioner calculate the distances, based on a general principle of anatomy. Spinal columns 6 in the strictest sense of human beings are almost all alike and related by a homothetic ratio (i.e. only their size changes). Thus, the distance separating a pair of typical vertebrae 10 divided by the total length of the spinal column 6 of the subject 15 remains constant in the human population (it does not depend on the subject 15). For example, as illustrated in FIG. 9, the distance between the vertebra C2 and the vertebra T3 is 1 and the total length of the spinal column in the strictest sense 6 is L. the ratio 1/L does not depend on the subject 15 in question. The device 14 is therefore configured to comprise a register containing this ratio for each pair of typical vertebrae 10. This register may form an integral part of the device 14. It is in practice recorded in the memory of the second processing unit.

The distance between two typical vertebrae 10 of a subject 15 may then be calculated by multiplying the total length of her or his spinal column 6, which will have been measured before the examination, and the ratio contained in the register between the distance separating these two typical vertebrae 10 and the total length of the spinal column 6.

This anatomical method has its limitations, however. It is unsuitable for example in the pathological case of a vertebral compression fracture. It does not allow the distance between the head 8 and typical vertebrae 10 to be calculated either. It is therefore important for the practitioner to be able to modify the distances between the vertebrae, including the head 8, so that it corresponds well to the ground truth that she or he observes.

The distances thus calculated or measured are recorded in a table of distances. The table of distances is recorded in the memory of the second processing unit 30.

The device 14 is configured to allow the practitioner to input the length of the spinal column 6, to modify the table of distances, to take into account radiographic documents, etc.

It is also necessary, prior to using the device 14, to correct the orientation of the inertial measurement units 16.

The notion of correction will be better understood by giving an example. An inertial measurement unit 16 placed on a typical vertebra 10 is supposed to be placed at the end of the spinous process 11 of said typical vertebra 10. In fact, it is difficult to place it exactly in the desired location because of the tip at the end of the spinous process 11 and because of the skin 13, which hides the typical vertebra 10 and which “plays” mechanically.

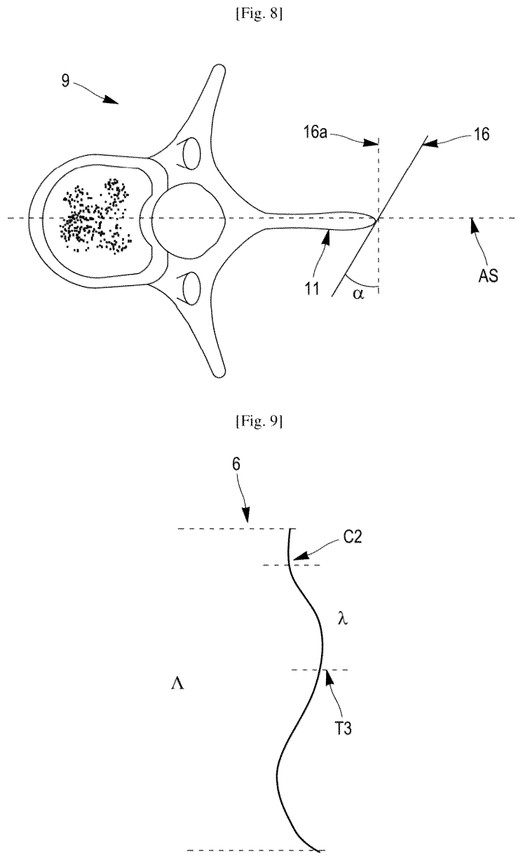

FIG. 8 illustrates the case of an inertial measurement unit 16 placed at an angle on the spinous process 11 of a typical vertebra 10 (the skin 13 between the inertial measurement unit and the typical vertebra 10 has not been shown). The unit 16, the typical vertebra 10 and spinous process 11 have been shown from above in cross-section in a transverse plane. Thus, the edge of the inertial measurement unit 16 makes an angle (-a) to the position 16a that it was supposed to have. In other words, to amend an orientation measurement taken by the inertial measurement unit 16 to make it match an orientation of the typical vertebra 10, it must be corrected by applying thereto a rotation of angle a about the axis perpendicular to the plane of the figure.

By “correct the orientation”, what is meant is precisely calculate the correction rotation separating the orientation of a typical vertebra 10 from the orientation of the respective inertial measurement unit 16 at the beginning of a measurement sequence. Let V be a trihedron representing the typical vertebra 10, let C be a trihedron representing the respective inertial measurement unit 16 at the beginning of the measurement sequence and let R be the correction rotation. Therefore, V=RC. This principle is generalizable to the head 8.

There are various methods for calculating R. For example, in a method that uses the principles of biomechanics, the subject is instructed to look horizontally by staring at a target. In this posture, the vertebra has a defined position and hence the term V of the above equation is known, the term C being known from measurements taken by the inertial measurement unit 16, and R being deduced therefrom. This calculation is performed by the second processing unit 30.

In the present application, a typical vertebra 10 is approximated as a non- deformable solid, and hence the correction rotation R remains constant throughout a measurement sequence. Therefore, during any measurement interval, the orientation of a typical vertebra 10 may be accurately deduced from the orientation of the respective inertial measurement unit 16 by multiplying it by R.

The correction function is also useful when an inertial measurement unit 16 is placed on the head 8 of the extended spinal column 9. In fact, this function makes it possible to place the inertial measurement unit 16 anywhere on the head 8. As the head 8 is a non-deformable solid, the reasoning followed in respect of the typical vertebrae 10 applies and it is possible to deduce, in any measurement interval, the position of the head 8 from the position of the inertial measurement unit 16 by applying thereto the initially calculated correction rotation R.

Another advantage of being able to correct the orientation of an inertial measurement unit 16 is that it relieves the practitioner of the need to do the very precise work required to place the inertial measurement units 16 in the same place, in each of the exercise sessions of the subject 15.

The device 14 is configured to allow the practitioner to modify the correction rotations R depending on her or his observations.

It is also necessary, prior to using the device 14, to adjust the inertial measurement units 16 in such a way that they deliver the same orientation measurements when they are placed in the same way. Specifically, an inertial measurement unit 16 is supposed to indicate an absolute direction, which is the Earth's magnetic north, by virtue of a magnetometer. In fact, the inertial measurement unit 16 diverges in the sense that it does not exactly indicate the Earth's magnetic north. It is therefore important to correct this divergence. In the device of the invention, it is not divergence with respect to the Earth's magnetic north that is corrected, but rather divergence with respect to a reference direction common to all the inertial measurement units attached by the user. A divergence correction rotation is therefore measured for each inertial measurement unit 16 and recorded in the second processing unit. The divergence correction rotation converts the reference direction into North as indicated by the inertial measurement unit. When an orientation derived from the measurements of the inertial measurement unit 16 reaches the second processing unit 30, it corrects the orientation via right composition by the respective divergence correction rotation and thus an orientation of the inertial measurement unit 16 with respect to the reference direction is obtained.

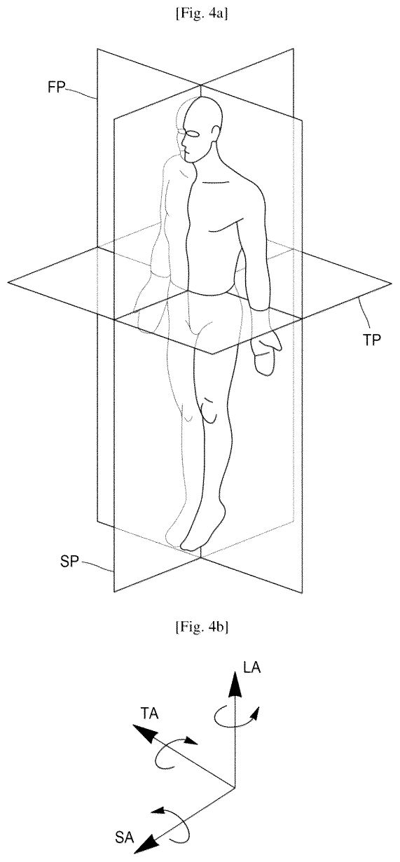

To clearly describe the measurements and the processing performed on the measurements, a few geometric notions and notations will be introduced. In anatomy, a human being is observed in three so-called “anatomical” planes as in FIG. 4a: the plane seen from front or back, called the frontal plane FP; the plane seen from right or left, called the sagittal plane SP; and the plane perpendicular to the other two, called the transverse plane TP. Anatomical axes are also introduced, in relation to the anatomical planes, and oriented as in FIG. 4b: the longitudinal axis LA from feet to head; the sagittal axis SA from rear to front; and the transverse axis TA from left to right.

Furthermore, three rotational movements are introduced about the anatomical axes: roll in the counter-clockwise direction about SA; pitch in the counter-clockwise direction about TA; and yaw in the counter-clockwise direction about LA, as shown in FIG. 4b. According to a theorem due to the mathematician Euler, it is possible to describe any rotation in the system of anatomical axes by composition of a roll, of a pitch and of a yaw taken in this order. This description of rotations is used in navigation, aeronautics, and robotics. The present invention applies it to the biomechanics of the spinal column.

During a measurement cycle, each inertial measurement unit 16 measures the velocities of rotation about its own system of axes and quantifies the measurements. The measurements are then processed by the first processing unit 20 associated with the inertial measurement unit 16. According to a conventional method, the velocities of rotation are integrated over the time interval in which they were measured, to give angles of rotation about the axes of the inertial measurement unit 16. Roll, pitch and yaw angles and therefore the rotation made by the inertial measurement unit 16 during the time interval, and therefore its orientation at the end of the time interval, are deduced thereby.

The inertial measurement units are for example based on micro-electro- mechanical systems (MEMS). Each inertial measurement unit is mounted on a circuit board with a microcontroller to which computation of orientations is entrusted. The microcontroller is advantageously programmed with a Kalman filter that is used, as Kalman filters conventionally are, to correct a measurement taken by comparing it with a prediction made based on past measurements and on the measurement taken. In the present case, the two main sources of errors in the measurements are due to: sudden movements of the back, which interfere with the movement requested by the practitioner; and drifts in the signals measured by the electronic components of the inertial measurement units 16.

In another variant (not shown in the drawings) the inertial measurement units 16 are connected to a “mother” circuit board worn on the belt of the subject. In this variant, there is a single first processing unit 20 common to the inertial measurement units 16, which processing unit is implemented in the mother circuit board.

The orientations of the inertial measurement units 16 are then transmitted to the second processing unit 30. They are completed, for each inertial measurement unit 16 and each respective vertebra, by a datum expressing the distance of the vertebra from a reference vertebra, for example the sacrum 2. A position of the vertebra and hence a position of the plurality of vertebrae is thus determined.

The second processing unit 30 interpolates a curve segment 32 between two successive inertial measurement units 16 based on the orientation of the first of the two measurement units and on the distance separating them.

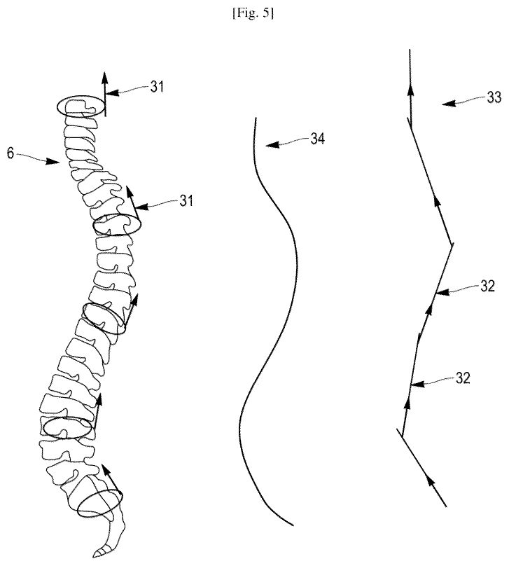

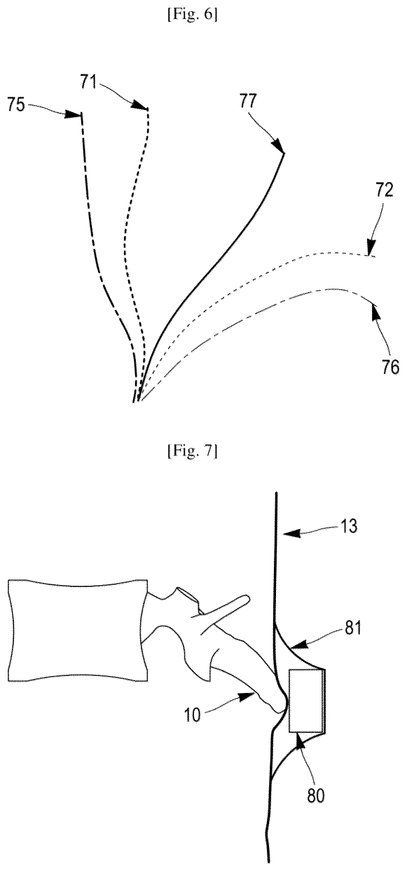

An interpolation method, illustrated in FIG. 5, allowing a first approximation consists in projecting the orientation of the inertial measurement units 16 in question into the frontal and sagittal planes and in considering each vector 31 thus obtained to be a direction vector of the tangent to the extended spinal column 9 (in FIG. 5, only the spinal column 6 has been shown) in the plane in question, level with the vertebra facing the inertial measurement unit 16 in question. The tangents intersect and thus form a polygonal chain 33. The greater the number of inertial measurement units, the greater number of measurement points, and the more the polygonal chain looks like a curve 34.

A more realistic representation of the extended spinal column 9 may be obtained by using curve-building methods such as the Euler method or the Runge-Kutta method.

Thus, in each measurement time interval, a set of geometrical points is obtained, this set of geometrical points, which form the curve 34, being on the one hand a representation of the measurements and on the other hand calculated, i.e. interpolated. The calculation of geometric points, for interpolation purposes, makes it possible to considerably reduce the number of geometric points to be measured.

The curve 34 represents the extended spinal column 9. After the calculations, the second processing unit 30 verifies whether the curve 34 respects biomechanical limits of the extended spinal column 9. For example, it verifies that the angular deviation between two successive typical vertebrae 10a and 10b (illustrated in FIG. 11) does not exceed a certain limit. If it exceeds this limit, the practitioner is notified by a message from the device that may for example take the form of a window displayed on the display unit 50 and containing an error message. It is up to the practitioner to analyze this anomaly. In practice, it is very often due to inaccuracies in the measurements of the inertial measurement units 16 or to erroneous input data. It may then be necessary, according to the circumstances, to “reset” the inertial measurement units 16 or correct the input data.

The device 14 has been designed for a moving subject 15 but is entirely suitable for the important clinical case of a still subject 15.

Images of the spinal column, which are formed from sets of geometric points, are then displayed on the one or more display units 50. In addition, it is possible, using known computer-graphic-generating methods, such as those used in the technical fields of computer aided design, to give the images a realistic appearance mimicking that of the spinal column 6/9, to make them appear as though they are in relief, to make the images rotate, to see the spinal column 6/9 from various angles, etc. (reference then being made to “avatar mode” or “3D mode”). A representation taking the form of a polygonal chain connecting the sensors is also advantageous for the practitioner (this is the “geometric mode”). If a whole measurement sequence has been taken, the image sequence may also be scrolled through to see the movement made. These images are meaningful to the practitioner and help her or him to assess the condition and behavior of the back of the subject 15 during a movement.

The device operates “in real time” in the sense that between two successive cycles of measurements taken by the inertial measurement units 16, the device 14 completes one complete processing cycle, in the first and second processing units 20, 30, resulting in display of the curve 34 representative of the spinal column (optionally in a form more elaborate than the curve 34) on a display unit 50. Thus, the device 14 updates the positions of the plurality of vertebrae and displays them in the desired graphical form before the next measurement. The processing and display time must be compatible with a measurement acquisition frequency of at least 12 Hz. It is obviously compatible with lower frequencies.

Display and/or recording of the positions of the plurality of vertebrae are/is triggered by the practitioner employing the device 14 (only the positions of the plurality of vertebrae are recorded, not the curve formed therefrom). The practitioner may display images and/or record positions of the plurality of vertebrae one at a time or in sequence. If she or he chooses to record in sequence, she or he may adjust the frequency of the recordings.

Subsequently, the practitioner may “replay” a recorded sequence. The second processing unit will again form curves based on the positions of pluralities of vertebrae. When she or he replays the recorded sequence, the practitioner may remove certain positions from the plurality of vertebrae if she or he deems them unuseful.

Every adjustment that the practitioner has to make and every command that the practitioner has to send to the device 14, in particular those mentioned (table of distances, biomechanical limits, anatomical data of the subject, frequency of acquisition of the measurements), are made/sent through a human-machine interface.

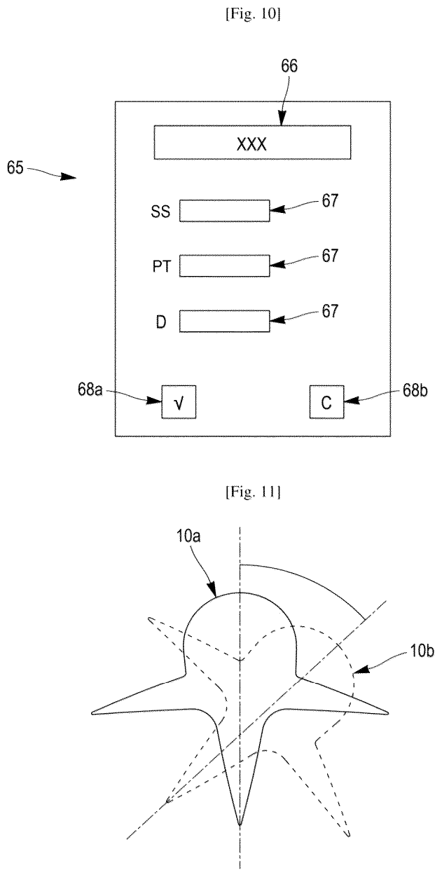

The human-machine interface uses, in every embodiment, at least one interface display unit 60, as shown in FIG. 1. In general, the interface display unit 60 is integrated into the display unit 50. Conventionally, the interface display unit 60 displays windows 65 as in FIG. 10 with: a title zone 66 indicating for example “Anatomical information”; boxes to be filled in 67, requesting input of “Sacral Slope” SS, “Pelvic Tilt” PT, and “Distance” D; and a validation button 68a and a cancellation button 68b. The window 65 disappears when one of the buttons is pressed. The interface display unit 60 makes it possible to “navigate” between pages via links or cross-references etc. The interface display unit 60 for example takes the form of a touch screen with an integrated keyboard, i.e. a keyboard displayed on the interface display unit 60. The touch screen means the practitioner does not need to employ untidy peripherals such as a keyboard and mouse. A display unit of the human-machine interface 60 may be integrated into a fixed terminal intended to be installed near an exercise station of the subject 15.

In one preferred embodiment, the human-machine interface comprises a “hands-free” control unit taking the form of a double pedal or of a system based on voice commands. The first pedal is used to record the positions of the plurality of vertebrae, and the second pedal is used to stop recording. The voice commands perform the same functions; each function corresponds to one agreed word or short succession of words.

In one embodiment, positions of a second plurality of vertebrae 71, 72, 75, 76, intended to be compared with positions of the plurality of vertebrae that are being captured by means of the device 14 or intended to be compared with one another, may be retrieved from the database 40.

We will now describe the three main use cases: exercise sessions, assessment and comparison.

As regards exercise sessions, it is a question of the case where a person, subject 15, has been instructed by a medical practitioner to perform rehabilitation exercises.

The practitioner starts an exercise session through the human-machine interface. A window called “Sensor arrangement” appears on the interface display unit 60. A new selection of measurement points may be created among the vertebrae 10, including the head 8. In this case, the practitioner installs the patches 81 and then the inertial measurement units 16 one after another in the locations she or he has selected and indicates facing which bone element she or he has placed them. She or he measures the distance between each sensor and records it in the second processing unit 30.

A list may be selected from and a pre-recorded selection of measurement points used. For example, selection of measurement points using 7 inertial measurement units 16 arranged on the head 8 and then on vertebrae C7, T4, T9, T12, L3, S2 in general achieves the best compromise between the accuracy of the observations and the volume of data, when it is a question of observing the entire spinal column. It is then necessary to place patches 81 and inertial measurement units 16 according to the selection. This is done either using the given distances, or by placing the inertial measurement units 16 facing the corresponding vertebrae. The practitioner validates this step.

A new window appears on the interface display unit 60. The bulk of this window displays a representation of the moving extended spinal column 9, in real time and in 3D. To the side, 3 small windows make it possible to see views in the 3 anatomical planes SP, FP, TP. The principal center of rotation 9 is exterior to the sacrum 2.

The practitioner may improve the realism of the curve to get closer to what she or he considers to be the ground truth, by correcting the angles given by the inertial measurement units 16. She or he opens an “angular adjustment” function and is able to modify, in each plane, the angulation of each inertial measurement unit 16. It is a question of a simplified case of application of a correction rotation, as illustrated in FIG. 11. The device 14 is also able to provide assistance. For example, in the case of the head 8, the subject 15 is asked to look straight ahead and to validate that she or he considers her or his head 8 to be straight. According to another example, in the case of the sacrum 2 and of the pelvis in general, the device of the invention takes into account anatomical data already recorded, and in particular sacral slope and lateral inclination. The result may be checked and corrected. The practitioner is also able to modify the distance between the inertial measurement units 16 by returning to the “Sensor arrangement” window. She or he may thus improve the representation of the pelvis by correcting the distances and angulations between the sacrum and the hip joints, or indeed validate use of pre-recorded anatomical data. Once she or he is satisfied with the representation of the extended spinal column 9, she or he may ask the subject 15 to adopt a posture that is easy to reproduce, and record the form of the corresponding representation.

Throughout the session, a light notifies her or him of the adequateness of the data used. In case of an amber or red light, she or he may return to the “Sensor status” window or “Sensor arrangement” window, according to the circumstances. She or he may thus either, for example, re-inspect lost inertial measurement units 16, or reset them in a recorded position, or replace an inertial measurement unit 16 with a spare inertial measurement unit 16. She or he may, at any time, return to the “Sensor arrangement” window and modify their locations and number, with a view to a broader or more localized examination.

The practitioner may use the device to make an “assessment”.

During an assessment, the practitioner instructs the subject 15 to make a movement. She or he records, during the movement, the positions of the plurality of vertebrae that she or he deems relevant and crucial. She or he uses a double foot pedal or a voice command to free her or his hands and also allow her or him to move around the patient.

Once the positions of the plurality of intermediate vertebrae deemed important have been recorded, she or he ends the recording and opens a new window displaying, at its center, a reconstruction of the recorded positions, which reconstruction is animatable. She or he may view this animation “in 3D”, in “geometric mode” or “avatar mode”, with or without vectors, etc., and in the anatomical planes SP, FP, TP. She or he may remove positions deemed unuseful, record photos that seem illustrative to her or him for a report, and/or add annotations. These functions are particularly useful when preparing reports (as often required in medical practice) in the form of computer files.

The comparison function makes it possible to compare a medical examination of the subject 15 with a previous medical examination recorded in the database. An examination is essentially characterized by a movement to be made and by a selection of measurement points.

Before starting a comparison, the practitioner displays the records of the subject 15 on the display unit of the human-machine interface 60. She or he then chooses from the records the examination to be repeated. The selection of measurement points is mentioned therein. She or he places the patches 81 and inertial measurement units 16 as indicated on the selection of measurement points.

The practitioner then starts the comparison and instructs the patient to make the movement of the examination. A specific window displays positions of a second plurality of vertebrae 71, 72, 75, 76 retrieved from the database and producing an animation of the movement made during the last examination, and highlighted the positions of the first plurality of vertebrae corresponding to the movement that the subject 15 is in the process of making.

The practitioner also starts the recording and the second processing unit 30 automatically records the positions of the first plurality of vertebrae when they are in a vicinity of the positions of the second plurality of vertebrae. The notion of vicinity depends on the application of the device 14 (spinal column, lower limb, upper limb, etc.). It also depends on the selected measurement points. For example, in the case of the spinal column, assuming that the sacrum 2 forms part of the selected measurement points, the term “vicinity” may designate an interval of values of the anatomical parameter “sacral slope”.

Once the subject 15 has finished making their movement, the interface display unit 60 opens a window in which the first and the positions of the second plurality of vertebrae 71, 72, 75, 76 recorded are superposed. This function allows the practitioner to analyze differences between the positions of the first and second plurality of vertebrae. She or he may analyze the difference by modifying the viewing angle or selecting the thumbnails of the anatomical planes SP, FP, TP. She or he may, by virtue of a software tool, measure angles she or he deems useful. She or he may prepare a report as often required in medical practice, taking the form of a computer file.

To compare a plurality of recordings with one another, i.e. a plurality of positions of the second plurality of mutually generalized vertebrae, 71 and 75 for example, the practitioner merely displays the positions of the second plurality of vertebrae on the display unit 50 and makes a visual comparison.

In the most general embodiment of the device 14 of the invention, in which it is suitable for types of continuous successions of bone elements other than the extended spinal column 9, the device 14 allows the practitioner to choose, from the outset, the continuous succession of bone elements 9 of interest to her or him, one of the lower limbs for example. The practitioner may also modify the distances and angulations between the sacrum 2 and the hip joint 7, possibly based on pre-recorded anatomical data such as an x-ray, to improve in her or his eyes the representation of the pelvis.

In the example of one of the lower limbs, a selection of relevant measurement points is formed from 5 inertial measurement units 16, one placed facing the sacrum 2, two placed facing the 2 trochanters, one placed facing the head of the fibula, and one placed on the back of the foot (the entire foot being considered to be one bone element). For a lower limb, there is then a center of rotation 7 level with the hip joint 7.

It will be noted that the first processing unit 20 and the second processing unit 30 may be produced independently in various forms, in a unitary or distributed manner, by means of hardware and/or software components. Usable hardware components are application-specific integrated circuits (ASICs), field-programmable gate arrays (FPGAs) or microprocessors. Software components may be written in various programming languages, for example C, C++, Java, or VHDL. This list is not exhaustive.

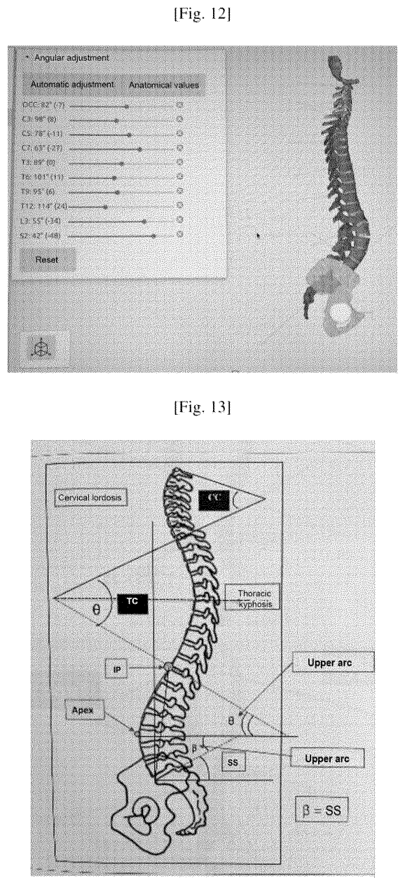

FIG. 12 illustrates one example of angular correction applied to the angular data generated by the sensors. These corrections are stored and taken into account in the calculations of the model, as described above.

FIG. 13 illustrates an example of angular corrections based on a theoretical postural model established beforehand, as described above.

Although the invention has been described in relation to a number of specific embodiments, it is evidently in no way limited thereto and it includes any technical equivalents of the means described and combinations thereof where these fall within the scope of the invention.

Use of the verb “comprise” or “include” or “have”, including when conjugated, does not exclude the presence of other clements or other steps in addition to those mentioned in a claim.

In the claims, any reference signs between parentheses should not be interpreted as a limitation to the claim.

1. A device for generating an image sequence, said device being configured to represent, in the three dimensions of space, at least one continuous succession of aligned bone elements of a skeleton, said at least one succession being able to be moving, the skeleton belonging to a subject, the device comprising:
a plurality of inertial measurement units,
each inertial measurement unit being configured to cyclically measure velocities of rotation about three axes associated with the inertial measurement unit, thus forming measurement data,
the inertial measurement units being intended to be placed facing selected measurement points on the succession of bone elements, in an amount of one inertial measurement unit per measurement point, said measurement points thus forming a plurality of measurement points to be ordered,
at least one first processing unit, configured to:
receive the measurement data of the plurality of inertial measurement units,
upon receipt of new measurement data, update based on said measurement data an orientation of each of the inertial measurement units of the plurality of inertial measurement units
and a second processing unit, configured to
receive the orientations of inertial measurement units-from the at least one first processing unit,
upon receipt of new orientations of inertial measurement units,
for each measurement point of the plurality of measurement points, update:
an orientation, based on the received orientations of the inertial measurement units
except for the first measurement point, a position, based on the orientation of the updated previous measurement point and on a table of distances between the bone elements of the succession of bone elements;
the position of the first measurement point being determined based on an axis of rotation of the continuous succession of bone elements,
re-interpolate, for each pair of successive measurement points of the plurality of measurement points, between the measurement points of said pair, a curve segment;
re-form a curve by joining the curve segments.

2. The device for generating an image sequence as claimed in claim 1, wherein the second processing unit is configured to, for each inertial measurement unit, indicating a direction assumed to be Earth's magnetic north,
calculate a divergence correction rotation, said divergence correction rotation converting a reference direction common to the inertial measurement units into the direction indicated by the inertial measurement unit;
when the orientation of the inertial measurement unit is updated, amend the orientation of the inertial measurement unit via right composition by the respective divergence correction rotation.

3. The device for generating an image sequence as claimed in claim 1,configured to represent, in the three dimensions of space, a moving spinal column fiss belonging to a subjects, the device comprising:
a plurality of inertial measurement units,
each inertial measurement unit being configured to cyclically measure velocities of rotation about three axes associated with the inertial measurement unit, thus forming measurement data,
the inertial measurement units being intended to be placed facing selected vertebrae of the spinal column, in an amount of one inertial measurement unit-per vertebra, said vertebrae thus forming a plurality of vertebrae to be ordered
at least one first processing unit, configured to
receive the measurement data of the one or more inertial measurement units, respectively,
upon receipt of new measurement data, update based on said measurement data, an orientation of each of the inertial measurement units of the plurality of inertial measurement units,
and a second processing unit, configured to
receive the orientations of inertial measurement units from the at least one first processing unit,
upon receipt of new orientations of the inertial measurement units,
for each vertebra of the plurality of vertebrae, update a vertebral orientation, based on the received orientations of the inertial measurement units,
except for the first vertebra, a position, based on the vertebral orientation of the updated previous vertebra and on a table of distances between the vertebrae of the spinal column;
the position of the first vertebra being determined based on an axis of rotation of the continuous succession of bone elements, thus forming a position of the plurality of vertebrae;
re-interpolate, for each pair of successive vertebrae of the plurality of vertebrae, between the vertebrae of said pair, a curve segment;
re-form a curve by joining the curve segments.

4. The device for generating an image sequence as claimed in claim 3:
having available to it a register that contains, for each pair of vertebrae of the spinal column of the subject, a ratio of the distance of said vertebrae to the length of the spinal column of the subject,
for a pair of vertebrae of the spinal column of the subject, the distance separating the vertebrae of said pair being calculated by multiplying the corresponding ratio of the register by the length of the spinal column ss of the subject,
said distance being automatically entered into the table of distances.

5. The device for generating an image sequence as claimed in claim 1, wherein the second processing unit is configured to allow a user to modify the table of distances.

6. The device for generating an image sequence as claimed in claim 5, wherein the second processing unit is configured to:
for each vertebra of the plurality of vertebrae and/or for the head, when an inertial measurement unit with an inertial measurement unit orientation is placed on the respective vertebra or head, which have a vertebral orientation, calculate a position correction rotation, the position correction rotation converting the orientation of the inertial measurement unit into a vertebral orientation of the respective vertebra or head;
upon receipt of new measurement data, for each vertebra of the plurality of vertebrae and/or for the head, prior to updating the orientations of vertebrae and/or the head, correct the orientation of the respective inertial measurement unit via left composition by the respective correction rotation.

7. The device for generating an image sequence as claimed in claim 6, wherein the second processing unit is configured to allow a user to modify the position correction rotations of the vertebrae.

8. The device for generating an animated image sequence as claimed in claim 1, further comprising a database and/or a display unit, and wherein the second processing unit is configured to allow a user to trigger, in each measurement cycle:
recording of the position of the plurality of vertebrae then formed in the databas;
and/or display of the curve then formed on the display unit.

9. The device for generating an image sequence as claimed in claim 8, wherein the measurement frequency is adjustable by a user.

10. The device for generating an image sequence as claimed in claim 8, wherein the second processing unitis further configured to:
allow a user to retrieve from the database at least one position of a second plurality of vertebrae; and, for each position of the second plurality of vertebrae:
interpolate between the vertebrae of each pair of successive vertebrae of the second plurality of vertebrae, a second curve segment; and
form a respective second curve by joining the second curve segments;
and in addition configured to display the second curves on the display unit.

11. The device for generating an image sequence as claimed in claim 10, wherein the second processing unit & is further configured to:
when the second processing unit receives new orientations, monitor whether the position of the updated plurality of vertebrae is in a vicinity of each position of the second plurality of vertebrae;
and, if so, generate a signal specific to each position of the second plurality of vertebrae.

12. The device for generating an image sequence as claimed in claim 1, wherein the second processing unit is equipped with a first pedal for recording the positions of the plurality of vertebrae, with a second pedal for stopping recording the positions of the plurality of vertebrae and/or with a voice command combining the functions of the first and second pedals.

13. The device for generating an image sequence as claimed in claim 1, further comprising a system for synchronously initializing the sensors or inertial measurement units.

14. The device for generating an image sequence as claimed in claim 1, being configured in such a way that:
the positions of the sensors are paired, the sensors being positioned freely on the patient at the discretion of the user and facing identified bone elements, these positions being communicated,
the calculation of the distances between two sensors, essential to correct modelling, is carried out in light of a pre-filled table collating the relative distances between each neighboring bone element,
the calculations of the modelling include the location of the sensors and the distance separating them,
in the event of a positioning level error, the latter is corrected by modifying this pairing.

15. The device for generating an image sequence as claimed in claim 1, being configured in such a way that an angular correction is applied to the angular data generated by the sensors, this correction being stored and taken into account in the calculations leading to the modelling.

16. The device for generating an image sequence as claimed in claim 1, being configured to:
communicate pelvic parameters resulting from radiology,
model the pelvis via integration into the calculations in view of reliable modelling of the pelvis according to anatomical parameters,
determine the instantaneous center of rotation of the sacrum; the real-time modelling potentially integrating rotation of the sacrum about the line between the center of rotation and the second sacral vertebra, as well as the movement of the entire suprajacent column as deduced from data generated by sensors arranged on the vertebrae, and
determine a reliable sacral slope angle in the erect position using an angular adjustment as seen above of the sensor arranged on the sacrum depending on the sacral slope calculated during a radiological examination in the same posture.

17. The device for generating an image sequence as claimed in claim 1, wherein a sensor arranged facing bone markings at each level generates angular information relating to bone elements of the lower limbs.

18. The device for generating an image sequence as claimed in claim 1, being configured to:
define one or more angles such that the one or more sides of an angle are defined relative to one or more bone elements taken into account in the modelling, with:
in the case where one side is relative to a bone element, the other side may be vertical or horizontal; if it is a question of representation of a vertebra, these angles correspond to angles of what is referred to as slope, when one of the sides is horizontal, and what is referred to as inclination, when one of the sides is vertical,
in the case where the two sides of an angle are defined relative to two bone elements taken into account in the modelling, the definition of this angle induces tracking of a modification of curvature of the set of bone elements included in this angle; it is thus in particular a question of tracking of modification of curvature of a set of vertebrae, for example angle of kyphosis, lordosis, scoliosis, the sides corresponding to the tangents of the superior endplate of the selected superior vertebra and the inferior endplate of the selected inferior vertebra,
for a defined angle, perform the calculation of this angle in three planes and display it in real time during the movement, and
collate the collected data relating to the angles.
